(19) United States
(12) Patent Application Publication
(10) Pub. No.: US 2010/0016305 A1
(43) Pub. Date: Jan. 21, 2010

(54) novel use of activators and stimulators of soluble guanylate cyclase for the prevention or treatment of renal disorders

(75) Inventors: Thomas Krahn, Hagen (DE); Johannes-Peter Stasch, Solingen (DE); Gerrit Weimann, Koln (DE); Wolfgang Thielemann, Wuppertal (DE); Matthias Rinke, Wulfrath (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Healthcare AG, Leverkusen (DE)

(21) Appl. No.: 11/989,068

(22) PCT Filed: Jul. 6, 2006
(86) PCT No.: PCT/EP2006/006601

§ 371 Date: Sep. 11, 2009

(30) Foreign Application Priority Data
Jul. 18, 2005 (EP) 05015522.5

(51) Int. Cl. A61K 31/5377; A61K 31/506; A61K 31/195; A61P 9/10

(52) U.S. Cl. 514/234.2; 514/256; 514/567

(57) ABSTRACT

The present invention relates generally to a method for the treatment of renal failure or renal hypertension and, more particularly, for improving the recovery from acute renal failure or renal hypertension by treatment with activators of soluble guanylate cyclase or stimulators of guanylate cyclase.

### FIELD OF INVENTION

The present invention relates generally to a production of a medicament for the treatment of renal failure or renal hypertension and, more particularly, to a production of a medicament for improving the recovery from acute renal failure or renal hypertension by treatment with activators of soluble guanylate cyclase or stimulators of guanylate cyclase.

### BACKGROUND OF THE INVENTION

The mammalian renal system serves primary roles both in the removal of catabolic waste products from the blood-stream and in the maintenance of fluid and electrolyte balances in the body. Renal failures are, therefore, life-threatening conditions in which the build-up of catabolites and other toxins, and/or the development of significant imbalances in electrolytes or fluids, may lead to the failure of other major organs systems and death. As a general matter, renal failure is classified as “acute” or “chronic”. As detailed below, chronic renal failure is a debilitating and life-threatening disease for which no adequate treatment exists.

Renal failure is a condition characterized by decreased number of functional nephrons, resulting in reduced excretion of nitrogenous metabolic products and eventually causing the failure to maintain homeostasis in the biological environment. Specifically, this can be said to be a condition in which blood urea nitrogen and creatinine levels are continuously increased. Renal failure is categorized into two primary types: acute renal failure and chronic renal failure which is slowly progressive but irreversible.

Acute renal failure is primarily categorized into the following two types: oliguric acute renal failure which is frequently complicated by water, electrolyte and acid-base imbalances and manifested by oliguria or anuria; and non-oliguric acute renal failure in which decreased urinary volume is not found.

Acute renal failure is also categorized into the following three types according to its cause:
1) pronephric acute renal failure in which reduction of renal blood flow occurs due to systemic hemodynamic changes such as prerenal dehydration and shock, causing reduced glomerular filtration rate
2) renal acute renal failure which is induced by glomerular and tubular-disorders such as acute tubular necrosis; and
3) postrenal acute renal failure which is caused by obstruction of the urinary tract, e.g. by a calculus.

According to the clinical manifestations, it can also be categorized into oliguric, uretic and recovery stages. In the treatment of acute renal failure, it is important to track down its cause and sufficiently perform systemic control of the patient. Such treatment includes two major forms, conservative treatment and dialytic treatment. According to the conservative treatment, in the oliguric stage, excessive water drinking is avoided and the amount of protein intake is restricted, while simultaneously supplying a sufficient amount of calories. In the oliguric stage, or when heart failure is occurred, then sodium intake is restricted. In contrast, in the uretic stage, potassium intake is increased.

Chronic renal failure is a condition in which gradual reduction in renal functions occurs due to a chronically progressive renal disease, in which the reduced renal functions are manifested as the insufficiency of all functions for which the normal kidney is responsible. The causal diseases of chronic renal failure are all of the nephropathic diseases, including primary renal diseases, congenital renal diseases, renal infections, nephropathy induced by any nephrotoxic substance and obstructive urinary disease. As seen in the clinical background of patients to whom dialysis has been introduced for treatment of chronic renal failure, the primary causal diseases of chronic renal failure may include chronic glomerulonephritis, diabetic nephropathy, chronic pyelonephritis, nephrosclerosis and cystic kidney. Among these, chronic glomerulonephritis and diabetic nephropathy make up a large proportion. The proportion of diabetic nephropathy as the causal disease in the total cases, however, remarkably increases as the number of diabetic patients rapidly increases in recent years.

As stated above, renal failure may be caused by various diseases. However, all types of renal failure have particular common clinical manifestations regardless of their causal diseases, such as hypertension, lung congestion and congestive heart failure associated with reduced urinary volume; neurological or mental complaints associated with advanced uremia; anemia caused by reduced production of erythropoietin in the kidney; electrolyte imbalance, such as hyponatremia and hyperkalemia; gastrointestinal complaints; defect of bone metabolism; and defect of carbohydrate metabolism.

The adaptations in early stage chronic renal failure are not successful in completely restoring glomerular filtration rate or other parameters of renal function and, in fact, subject the remaining nephrons to increased risk of loss.

For the treatment of chronic renal failure in the conservative stage, dietary therapy including a low-protein, high-calorie diet is basically employed. In this case, it is required to restrict sodium chloride intake and water intake and to use an antihypertensive agent to control the hypertension which may be a risk factor for exacerbation of renal failure. However, such dietary therapy and the treatment with an antihypertensive agent as mentioned above produce unsatisfactory effects. Therefore, the number of patients who inevitably have hemodialysis goes on increasing year by year due to the manifestation of uremic symptoms caused by the advanced disorders of renal functions. In patients with renal failure who have entered into dialysis, remarkable improvement in the rate of prolongation of life has been achieved due to the improved hemodialysis therapy in recent years. However, there still remain problems in that the patients are unavoidable to visit the hospital twice or three times a week that defects of erythrocyte production or maturation may occur.

The object of the present invention is to provide a therapeutic agent for renal failure and/or renal hypertension on which already-existing drugs or agents show unsatisfactory effects.

### DESCRIPTION OF THE INVENTION

The heterodimeric hemoprotein soluble guanylate cyclase (sGC) acts as the principal intracellular receptor for nitric oxide (NO) and facilitates the formation of the second messenger cyclic guanosine-3′,5′-monophosphate (cGMP), which in turn governs many aspects of cellular function via interaction with specific kinases, ion channels and phosphodiesterases. The signal transduction pathway underlies the majority of physiological actions attributed to NO and is important in the regulation of the cardiovascular, gastrointestinal, urogenital, nervous and immune systems. As a consequence, aberrant sGC-dependent signaling may be fundamental to the etiology of a wide variety of pathologies; agents that can modulate enzyme activity in a selective manner should therefore possess considerable therapeutic potential.

The use of organic nitrates (e.g. glyceryl trinitrate, GTN; isosorbide dinitrate) for the treatment of conditions such as angina and heart failure has been advocated for over a century, although the mechanism of action of such compounds was not elucidated until the late 1970s and found to involve metabolic conversion to NO and subsequent activation of sGC. Surprisingly perhaps, little attention has focused on the identification of selective sGC-modulating compounds particularly enzyme activators that are probably of greater interest therapeutically. This is despite the fact that sGC dysfunction is likely to have an equivalent impact on pathogenesis as inappropriate NO production and tissue-specific distribution of sGC isoforms may provide a means of targeting drug therapy.

Although clinicians have at their disposal organic nitrates (and other NO-donor or ‘nitrovasodilator’ drugs), which release the endogenous ligand NO to activate sGC, the use of such compounds is problematic. First, NO-donor compounds, particularly organic nitrates, suffer from the development tolerance following prolonged administration. The mechanism(s) underlying this tachyphylaxis remain unclear but may be linked to decreased metabolic activation of the compounds, excessive superoxide, endothelin or angiotensin II levels or a reduction in the sensitivity/activity of the NO receptor, sGC. Second, the use of NO-donors in vivo is potentially troublesome due to non-specific interaction of NO with other biological molecules; reactions that are difficult to control due to the spontaneous release of NO from nitrovasodilators and its free diffusion in biological systems. Current dogma suggests that the beneficial (physiological) actions of NO are mediated predominantly via activation of sGC (i.e. cGMP-dependent) and the detrimental (pathological) actions of NO are exerted primarily via direct (i.e. cGMP-independent) modifications of proteins (e.g. nitrosation, nitration), lipids (e.g. peroxidation) and nucleic acids (e.g. DNA strand breaks). Thus, user of NO-based therapeutics will always represent a double-edged sword. Even if doses are titred to minimize these side effects, the majority is not readily reversible and will accumulate over time, potentially manifesting as long-term problems. Moreover, persistent inhibition of oxidative phosphorylation by NO may trigger apoptosis and cell death. In light of these shortcomings, compounds which can activate sGC in an NO-independent manner, and not suffer from tachyphylaxis, will therefore offer a considerable advance on current therapy of cardiorenal diseases.

In recent years several NO-independent soluble guanylate cyclase activators have been identified. Based upon their characteristics, these compounds can be classified into two groups, the first comprising the NO-independent, but heme-dependent soluble guanylate cyclase stimulators such as compounds of the formula (I) to (II), and the second, the NO- and hemo independent soluble guanylate cyclase activators represented by compounds of the formula (IV) to (VI). The first group shows a strong synergism when combined with NO and a loss of effect after the removal of the prosthetic soluble guanylate cyclase heme moiety. In contrast, soluble guanylate cyclase activation by compounds of the formula (IV) is potentiated by the removal of the heme group due to high affinity binding sites for this compound including within the heme pocket of the apo-enzyme. The replacement of the heme group by the compound of formula (IV) can be strongly facilitated by oxidation of the heme moiety resulting in destabilization of the heme binding to the enzyme.

Examples of stimulators of soluble guanylate cyclase which may be mentioned are the compounds (I) to (III) according to the following formulas:

| | |
|---|---|
| 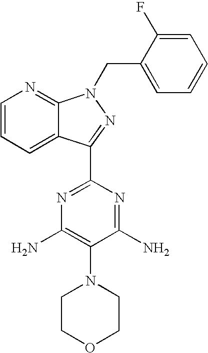 | Compound according to formula (I), its production and use as pharmaceutical active agent are disclosed in WO 00/06569. |
| (I) | |
| 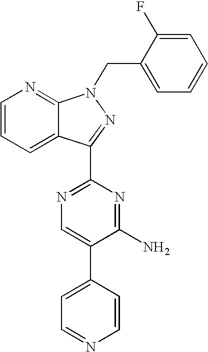 | Compound according to formula (II), its production and use as pharmaceutical active agent are disclosed in WO 00/06569 and WO 02/42301. |
| (II) | |
| 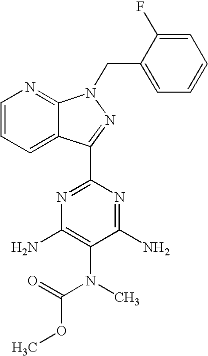 | Compound according to formula (III), its production and use as pharmaceutical active agent are disclosed in WO 00/06569 and WO 03/095451. |
| (III) | |
| 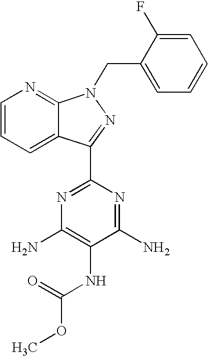 | Compound according to formula (IIIa), its production and use as pharmaceutical active agent are disclosed in WO 00/06569 and WO 03/095451. |
| (IIIa) | |

and the pharmacologically acceptable salts of these compounds.

Examples of activators of soluble guanylate cyclase which may be mentioned are compounds (IV) to (VI) according to the following formulas:

| | |
|---|---|
| 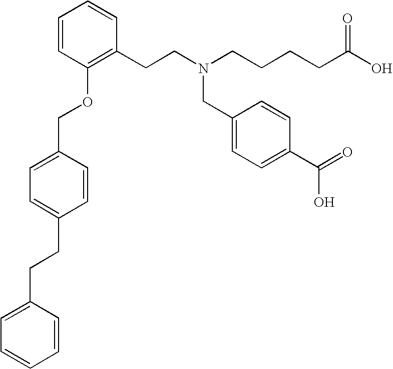 | Compound according to formula (IV), its production and use as pharmaceutical active agent are disclosed in WO 01/19780. |
| (I) | |
| 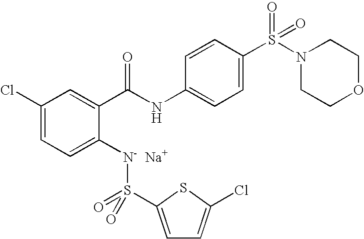 | Compound according to formula (V), its production and use as pharmaceutical active agent are disclosed in WO 00/02851. |
| (V) | |
| 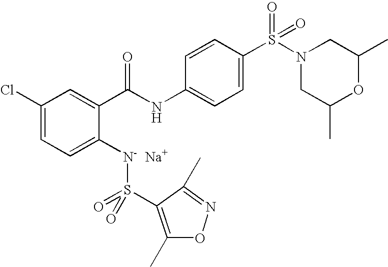 | Compound according to formula (VI), its production and use as pharmaceutical active agent are disclosed in WO 00/02851. |
| (VI). | |

and the pharmacologically acceptable salts of these compounds.

The method of the invention relates to administering to a subject an amount of sGC stimulators or sGC activators effective to reduce, inhibit or prevent symptoms of renal failure or renal hypertension in a mammal, including man. The administration can be enteral, e.g. oral or rectal; parenteral, e.g. intravenous; or transdermal.

As used herein the term “renal failure” means a disease state or condition wherein the renal tissues fail to perform their normal functions. Renal failure includes chronic and acute renal failure or dysfunction.

Acute renal failure is broadly defined as a rapid deterioration in renal function sufficient to result in accumulation of nitrogenous wastes in the body. The causes of such deterioration include renal hypoperfusion, obstructive uropathy, and intrinsic renal disease such as acute glomerulonephritis.

Chronic renal failure is usually caused by renal injuries of a more sustained nature which often lead to progressive destruction of nephron mass. Glomerulonephritis, tubulointerstitial diseases, diabetic nephropathy and nephrosclerosis are among the most common causes of chronic renal failure. Chronic renal failure can be defined as a progressive, permanent and significant reduction in glomerular filtration rate due to a significant and continuing loss of nephrons. The clinical syndrome that results from profound loss of renal function is called uremia.

Diagnostic signs of renal failure include lower than normal creatinine clearance; lower than normal free water clearance; higher than normal blood urea and/or nitrogen and/or potassium and/or creatinine levels; altered activity of kidney enzymes such as gamma glutanyl synthetase; altered urine osmolarity or volume; elevated levels of microalbuminuria or macroalbuminuria; glomerular and arteriolar lesions; tubular dilation; hyperphosphatemia; or need of dialysis.

The inhibition of the renal failure can be evaluated by measuring these parameters in mammals by methods well known in the art, e.g. by measuring creatinine clearance.

Renal failure can be divided into several stages starting from mild form followed by moderate and severe forms and processing to so called end stage renal disease. These stages can be identified in a conventional way e.g. by determining the creatinine clearance values for which well-defined ranges are assigned to the different stages of renal insufficiency.

The effective amount of sGC activators or sGC stimulators to be administered to a subject depends upon the condition to be treated, the route of administration, age, weight and the condition of the patient. In general, sGC stimulators or sGC activators are administered orally to man in daily doses from about 0.1 to 400 mg, preferably from 0.2 to 100 mg, more preferably from 0.5 to 20 mg, given once a day or divided into several doses a day, depending on the age, body weight and condition of the patient.

sGC stimulators or sGC activators can be administered by intravenous infusion using the infusion rate typically from about 0.01 to 10 μg/kg/min, more typically from about 0.02 to 5 μg/kg/min. For the intravenous treatment of renal failure an intravenous bolus of 10-200 μg/kg followed by infusion of 0.2-3 μg/kg/min may be needed.

sGC stimulators or sGC activators are formulated into dosage forms suitable for the treatment of renal failure and/or renal hypertension using the principles known in the art. It is given to a patient as such ore preferably in combination with suitable pharmaceutical excipients in the form of tablets, dragees, capsules, suppositories, emulsions, suspensions or solutions whereby the contents of active compound in the formulation is from about 0.5 to 100% per weight. Choosing suitable ingredients for the composition is a routine to those of ordinary skill in the art. It is evident that suitable carriers, solvents, gel forming ingredients, dispersion forming ingredients, antioxidants, colors, sweeteners, wetting compounds, release controlling components and other ingredients normally used in this field of technology may be also used.

Salts of sGC stimulators or sGC activators may be prepared by known methods as pharmaceutically acceptable salts.

### Experimental Methods

### 1. L-NAME Treated Renin Transpenic Rats (TGR(mRen2)27)

NO is synthesized in endothelial cells from L-arginine by NO synthase, which can be inhibited by L-arginine analogs such as L-NAME. Both acute and chronic inhibition of NO synthase worsens ischemic renal dysfunction and induces an increase in blood pressure in different rat strains and other experimental animals. In humans, vasodilatation by acetylcholine and bradykinin can be attenuated by infusion of an NO synthase inhibitor. The cardiovascular consequences of sGC stimulation and sGC activation were evaluated by determining the compound's long-term effects on hemodynamic and hormonal parameters in a high renin, low NO rat model of hypertension. In this study we used transgenic rats with an additional renin gene (TGR(mRen2)27) which represent a very sensitive model for the cardiovascular effects of compounds interacting with the NO/sGC system. Systolic blood pressure increase in old renin transgenic rats (TG-R(mRen2)27) receiving the NO synthase inhibitor L-NAME in the drinking water whereas in animals treated with both L-NAME and the sGC stimulator or sGC activator, this blood pressure increase can be prevented during the observation period. At the end of the study, renin activity, aldosterone, urea and creatinine in plasma can be used to show a kidney protective effect of sGC stimulators or sGC activators. The beneficial effects of sGC stimulators or sGC activators in this therapeutically relevant animal model can also be shown by a reduction in mortality.

### 2. 5/6 Nephrectomized Rats

A well established model of impaired kidney function are rats with 5/6 nephrectomy. These rats are characterized by glomerular hyperfiltration, development of progressive renal failure leading to end-stage kidney disease and hypertension induced left ventricular hypertrophy and cardiac fibrosis. Four groups are analyzed: a sham-operated control group, a 5/6 nephrectomized group, a 5/6 nephrectomized group treated with a sGC stimulator, a 5/6 nephrectomized group treated with a sGC activator. Rats are treated for about 12 weeks. The drugs are given orally by gavage. Renal insufficiency is induced in rats by 5/6 nephrectomy. This procedure involves complete removal of the right kidney followed two weeks later by ligation of upper and lower third of the remaining kidney. After the second surgery the rats develop progressive renal failure (GFR decreases) with proteinuria and hypertension. The heart is characterized by a uremic hypertensive heart disease. Without treatment rats die between week 16 and 26 due to end-stage kidney disease or hypertension induced end-organ damage.

Rats were being placed in metabolic cages for 24 hours for urine collection. Sodium, potassium, calcium, phosphate and protein will be determined. Serum concentrations of either glucose, CrP (only serum), ALAT (only serum), ASAT (only serum), potassium, sodium, calcium, phosphate, urea and creatinine were determined using the appropriate kits in an automatic analyzer. Protein concentration in urine and serum were measured with a pyrogallol red-molybdate complex reagent in a Hitachi 717 automated analyzer. Glomerular filtration rate was calculated by the endogenous creatinine clearance. Systolic blood pressure and heart rate were measured by tail-cuff plethysmography in conscious, lightly restrained rats. Body weight was measured weekly.

Plasma renin activity and urinary aldosterone were analyzed by a commercially available radioimmunoassay assay.

All rats were scarified at the end of the study. Blood was taken for measurement of routine clinical chemistry (glucose, crea, urea, liver enzymes, C-reactive peptide, sodium, serum-protein) and plasma renin activity. Body-, heart- and kidney-weight were measured.

Histological evaluation of heart and kidney were performed for evaluation of the protective cardiorenal effects of sGC stimulators and sGC activators.

1. A method for prophylaxis and/or treatment of renal failure or renal hypertension comprising administering an effective amount of a compound selected from compounds of formulas (I) to (VI) 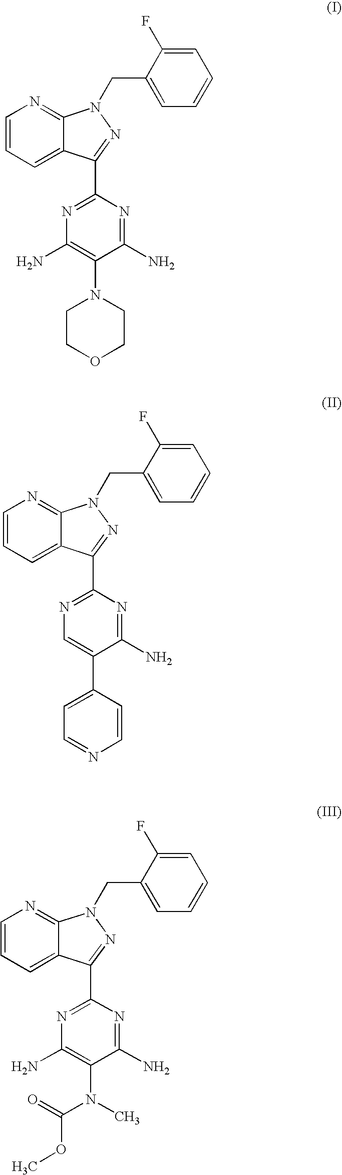 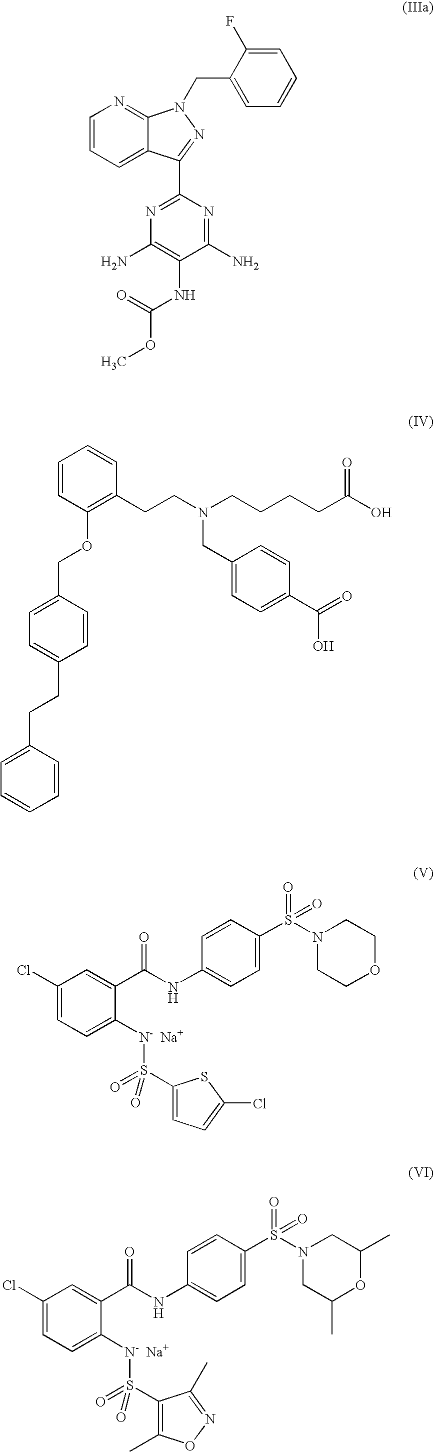
or a salt, hydrate, or hydrate of a salt thereof.

2. The method according to claim 1 for improving the recovery from acute renal failure or renal hypertension.

3. The method according to claim 1, wherein the medicament is for oral use compound is administered orally.

4. The method according to claim 1, wherein the compound is administered prophylactically.

5. A pharmaceutical composition for the treatment of renal failure or renal hypertension, comprising at least one compound of the formulas (I) to (VI), as defined in claim 1.
